(19) United States
(12) Patent Application Publication
(10) Pub. No.: US 2017/0175152 A1
(43) Pub. Date: Jun. 22, 2017

(54) MICROBIAL FERMENTATION OF ANHYDROSUGARS TO FATTY ACID ALKYL ESTERS

(71) Applicant: ExxonMobil Research and Engineering Company, Annandale, NJ (US)

(72) Inventors: Kelsey M. McNEELY, Lambertville, NJ (US); Virginia M. REINER, Summit, NJ (US)

(21) Appl. No.: 15/363,043

(22) Filed: Nov. 29, 2016

Related U.S. Application Data
(60) Provisional application No. 62/267,971, filed on Dec. 16, 2015.

(51) Int. Cl. C12P 7/64; C12N 9/00; C12N 9/02; C12N 9/10; C12N 9/16

(52) U.S. Cl. CPC C12P 7/6436; C12N 9/1029; C12N 9/16; C12N 9/0008; C12N 9/93; C12Y 203/01086; C12Y 301/02002; C12Y 102/0105; C12Y 602/01

(57) ABSTRACT

Methods are provided for biological conversion of anhydrosugars, such as anhydrosugars found in a pyrolysis oil, to fatty acid alkyl esters. The methods can include use of a genetically modified Escherichia coli (E. coli) bacteria that can convert levoglucosan and/or other anhydrosugars into fatty acid alkyl esters without requiring formation and conversion of an intermediate compound external to the bacteria. Optionally, the methods can be used in combination with methods for production and/or separation of increased amounts of levoglucosan from pyrolysis of biomass.

### CROSS-REFERENCE TO RELATED APPLICATIONS

This application claims priority to U.S. Provisional Application Ser. No. 62/267,971 filed on Dec. 16, 2015, herein incorporated by reference in its entirety.

### REFERENCE TO A SEQUENCE LISTING

This application contains references to amino acid sequences which have been submitted concurrently herewith as the sequence listing text file “2015EM405-US2.txt”, file size 4 KiloBytes (KB), created on 2 Feb. 2017. The aforementioned sequence listing is hereby incorporated by reference in its entirety pursuant to 37 C.F.R. §1.52(e)(5).

### FIELD

This invention relates to bio-conversion of anhydrosugars and/or other pyrolysis oil products into fatty acid alkyl esters.

### BACKGROUND

Developing renewable sources of feedstocks based on biomass for making distillate products, such as fuels or lubricants, is an area of ongoing interest. Use of biomass feedstock as a source of fuels is attractive from a perspective of reducing emissions associated with the use of transportation fuels. One source of biomass that is expected to have reduced emissions is lignocellulose. Lignocellulose is present in all plant biomass. Unfortunately, it has traditionally been difficult to convert lignocellulose to a desirable naphtha, diesel, or lubricant boiling range product.

One option for conversion of lignocellulose is to form a pyrolysis oil from a lignocellulose-containing feed. Pyrolysis can be effective for disrupting the bonds in lignocellulose to form potentially useful products, including anhydrosugars. However, further processing of pyrolysis oil (the product of pyrolysis) can typically be required in order to form a desired fuel product.

U.S. Patent Application Publication No. 2014/0024769 describes processes for making glycolic acid chemical intermediates and derivatives from biomass. The processes include heating a genetically modified polyhydroxyalkanoate (PHA) biomass to release glycolic acid monomers, which can then be used for further chemical synthesis. The biomass is described as being from a recombinant source that utilizes a carbon source, such as levoglucosan, as a feedstock.

U.S. Pat. No. 8,110,670 describes production of a variety of fatty acid derivatives based on fermentation of glucose by a variety of genetically modified microorganisms.

### SUMMARY

In an aspect, a method for converting levoglucosan to fatty acid alkyl esters, is provided, the method comprising: pyrolyzing a biomass feed under effective pyrolysis conditions to form a pyrolysis product comprising levoglucosan; performing a separation on at least a portion of the pyrolysis product to form a levoglucosan-enriched product; and culturing a recombinant Escherichia coli cell in the levoglucosan-enriched product to form a fermentation product comprising a fatty acid alkyl ester, the recombinant Escherichia coli cell comprising an expressed gene encoding a levoglucosan kinase enzyme and at least one expressed gene encoding a fatty acid derivative enzyme for production of the fatty acid alkyl ester internal to the Escherichia coli cell.

In another aspect, a cultured recombinant Escherichia coli cell is provided, said cell comprising: at least one expressed nucleic acid, operably linked to a first promoter that is constitutive, encoding an enzyme comprising an acyl-CoA synthase; at least one expressed nucleic acid, operably linked to a second promoter that is constitutive, encoding an enzyme comprising a thioesterase; at least one expressed nucleic acid, operably linked to a third promoter that is constitutive, encoding an enzyme comprising a fatty acyl-CoA reductase; and an expressed non-native nucleic acid, operably linked to a fourth promoter that is constitutive, encoding a levoglucosan kinase enzyme, wherein when cultured in the presence of a carbon source comprising levoglucosan, said cultured recombinant cell produces a fatty acid alkyl ester.

In still another aspect, a cell culturing environment is provided, comprising: at least about 0.1 wt % of a fatty acid alkyl ester; and at least about 0.1 wt % of cultured recombinant Escherichia coli cells, the cultured recombinant Escherichia coli cells comprising: at least one expressed nucleic acid, operably linked to a first promoter that is constitutive, encoding an enzyme comprising an acyl-CoA synthase; at least one expressed nucleic acid, operably linked to a second promoter that is constitutive, encoding an enzyme comprising a thioesterase; at least one expressed nucleic acid, operably linked to a third promoter that is constitutive, encoding an enzyme comprising a fatty acyl-CoA reductase; and an expressed non-native nucleic acid, operably linked to a fourth promoter that is constitutive, encoding a levoglucosan kinase enzyme, wherein the fatty acid alkyl ester comprises fatty acid alkyl ester produced by conversion of anhydrosugars by the cultured recombinant Escherichia coli cells

### BRIEF DESCRIPTION OF THE DRAWINGS

FIG. 1 schematically shows an example of a fermentation pathway in a recombinant E. coli cell for processing levoglucosan to form a fatty acid alkyl ester.

FIG. 2 shows an example of a protein sequence for levoglucosan kinase that can be formed by an E. coli cell based on introduction of a corresponding gene.

FIG. 3 schematically shows an example of a pyrolysis reaction system.

### DETAILED DESCRIPTION OF THE EMBODIMENTS

In various aspects, methods are provided for biological conversion of anhydrosugars, such as anhydrosugars found in a pyrolysis oil, to fatty acid alkyl esters. The methods can include use of a genetically modified Escherichia coli (E. coli) bacteria that can convert levoglucosan and/or other anhydrosugars into fatty acid alkyl esters without requiring formation and conversion of an intermediate compound. Optionally, the methods can be used in combination with methods for production and/or separation of increased amounts of levoglucosan from pyrolysis of biomass, although levoglucosan produced by alternate production and/or separation methods can be utilized by the genetically modified Escherichia coli (E. coli) bacteria according to the invention.

Lignocellulosic biomass is an abundant potential source of biomass for conversion into fuel. Lignocellulosic biomass can typically be present in substantially all plant-based biomass as part of the cellular structure. As a result, an efficient method for transforming lignocellulosic biomass to desirable fuel products could substantially increase the yield of products from plant-based biomass sources.

Unfortunately, lignocellulosic biomass can be difficult to convert to useful products. One current strategy can be to use pyrolysis to convert the lignocellulosic biomass to pyrolysis oil. Pyrolysis can be effective for conversion of biomass into other products without requiring the use of a catalyst and/or additional reagents such as hydrogen. The resulting pyrolysis oil can include a variety of compounds, including a high percentage of oxygenates and/or organic acids. At least a portion of the resulting pyrolysis oil can be further processed using conventional refinery processing techniques, such as hydroprocessing. However, due to the high percentage of oxygenates and organic acids, hydroprocessing of the pyrolysis oil can be costly, thus reducing the benefit of converting the pyrolysis oil to fuel products.

A desirable alternative to attempting to hydroprocess pyrolysis oil can be conversion of pyrolysis oil (or at least portions of the pyrolysis oil) using bacteria. At least some of the pyrolysis oil products can correspond to molecules suitable for conversion by bacteria, such as sugars. Some sugars can be readily converted to other products. However, the sugars present in pyrolysis oil can predominantly exist in the form of anhydrosugars, such as levoglucosan. Anhydrosugars can pose a difficulty for conversion via bacteria, as conventional bacteria are unable to directly convert anhydrosugars to a fuel product, such as a fatty acid alkyl ester, without first having a separate step of converting the levoglucosan to glucose by hydrolysis.

Levoglucosan (1,6-anhydro-β-D-glucopyranose) is the anhydrosugar of glucose. It can be produced during pyrolytic conversion of lignocellulosic biomass during which glucose from cellulose and/or hemicellulose can be dehydrated to levoglucosan. Anhydrosugar yields from pyrolysis of pure cellulose can typically be about 55 wt % to about 60wt %. Conventionally, fast pyrolysis of whole lignocellulosic feeds can result in levoglucosan yields of about 0.1 to about 10 wt % of the dry biomass. Levoglucosan yields can depend on the type of biomass as well as process conditions.

Levoglucosan yields can additionally or alternatively be limited due to competing reactions of carbohydrates and consecutive reactions of levoglucosan. For example, alkali and alkali earth metals often present in biomass can catalyze competing reactions and cause C—C bond cleavage of carbohydrates to lower molecular weight oxygenates, rather than dehydration to levoglucosan. Levoglucosan yields can be increased to up to approximately 12-30 wt % of the dry biomass, if the catalyzing metals can be removed by a thorough water wash and/or rendered catalytically inactive (passivation) prior to thermochemical conversion of the biomass. The latter can be achieved by treating the biomass with acids, such as hydrochloric acid, acetic acid, nitric acid, phosphoric acid and/or sulfuric acid, where the biomass can be mixed/impregnated with the aqueous acid solution and then dried. The amount of acid can be selected to correspond to the concentration of metals in the biomass, so that all metals can be converted to thermally stable salts and can advantageously not catalyze too much carbohydrate decomposition. While this type of acid treatment method can increases levoglucosan yields, it can have several drawbacks. One difficulty can be ensuring sufficient contacting of the biomass with the acid solution. In order to achieve a sufficient level of contact, the biomass can be sized to very small dimensions (e.g., <1 mm). Further, large amounts of water can be applied in the biomass pretreatment, which have to be removed prior to thermochemical processing. This can be done by filtration and drying of the biomass. Both sizing and water removal can be energy intensive and can contribute significantly to the economics and the carbon footprint of the process.

An additional difficulty with attempting to process pyrolysis oil can be the presence of compounds within the pyrolysis oil that can inhibit conversion by bacteria. For example, pyrolysis oil can typically include a variety ketones, aldehydes, and phenols. Such compounds can potentially act as poisons for some types of bacteria, such as E. coli. The presence of poisons in a feed for conversion by bacteria can reduce and/or minimize the effectiveness of the bacteria for conversion of the desired anhydrosugars.

Still another difficulty with attempting to process anhydrosugars can be the variety of anhydrosugars present in pyrolysis oil. The anhydrosugars in pyrolysis oil can correspond to both 5 carbon anhydrosugars and 6 carbon anhydrosugars. It is believed that yeast cannot process the 5 carbon anhydrosugars without genetic modification.

In various aspects, one or more of the above problems can be overcome by processing (fermenting) anhydrosugars derived from pyrolysis oil using genetically modified E. coli. The E. coli can be modified to be capable of conversion of levoglucosan without requiring prior chemical hydrolysis. The genetically modified E. coli can also be modified to produce fatty acid alkyl esters as a fermentation product. Such a fermentation product can optionally correspond to an excreted/secreted product, so that the fermentation product can be at least partially harvested without having to disrupt the cellular structure of the E. coli. Additionally or alternately, the genetically modified E. coli can be used to process anhydrosugars derived from pyrolysis oil having an enhanced content of levoglucosan and/or having been further processed to reduce and/or minimize the presence of poisons.

Levoglucosan can be one of many products formed during pyrolysis of lignocellulosic biomass. At least some of the additional pyrolysis products can behave as enzyme inhibitors and/or poisons to fermenting organisms. Thus, it can be beneficial to isolate and/or purify levoglucosan from the product stream prior to fermentation of the levoglucosan downstream. One method to isolate a product stream in which levoglucosan is concentrated can be by staging the condensation of product vapors. For example, during conventional biomass pyrolysis, the product vapors can be simultaneously quenched to produce a single pyrolysis product, namely pyrolysis oil. However, a controlled condensation at various selected temperatures can allow for the generation of a plurality of product fractions with corresponding boiling ranges. This method can allow for separation of heavier (higher boiling point) products, such as anhydrosugars, including levoglucosan and phenolic oligomers, from lighter products. The resulting heavy product stream containing levoglucosan and phenolic oligomers can then undergo further processing to isolate and/or enrich levoglucosan. For example, phenolic oligomers can be removed by washing the heavy product stream with water. Unlike levoglucosan, the phenolic oligomers are not typically highly soluble in water and can be removed, e.g., via filtration or centrifugation.

In some optional aspects, a controlled condensation can be performed using a separation device having sufficient separation power to provide a relatively narrow difference between a selected fractionation temperature and the actual final boiling point/initial boiling point of the respective fractions formed by the separation. An example of a fractionator and/or condenser for performing a separation with reduced and/or minimized overlap in the boiling ranges of the resulting fractions can be a distillation column having a separating efficiency equivalent to at least about 20 trays, for example at least about 30 trays, at least about 40 trays, or at least about 50 trays. In some aspects, a fractionation/condensation can be characterized based on the difference between the initial boiling point of the resulting higher boiling fraction and the final boiling point of the resulting lower boiling fraction. In such aspects, the difference between the initial boiling point of a higher boiling fraction and the final boiling point of a resulting lower boiling fraction can be about 40° F. (˜21° C.) or less, for example about 30° F. (˜17° C.) or less, about 25° F. (˜14° C.) or less, about 20° F. (˜11° C.) or less, about 15° F. (˜8° C.) or less, or about 10° F. (˜6° C.) or less. It is noted that this difference can typically correspond to a situation where the initial boiling point of the higher boiling fraction is less than the final boiling point of the lower boiling fraction by the indicated amount. In additional or alternative aspects, a fractionation can be characterized based on the difference between a T95 boiling point for the lower boiling fraction and the T5 boiling point for the higher boiling fraction. In such aspects, the difference between the T95 boiling point of the lower boiling fraction and the T5 boiling point of the higher boiling fraction can be about 40° F. (˜22° C.) or less, for example about 30° F. (˜17° C.) or less, about 25° F. (˜14° C.) or less, about 20° F. (˜11° C.) or less, about 15° F. (˜8° C.) or less, or about 10° F. (˜6° C.) or less. In some alternative aspects, the T5 boiling point for the higher boiling fraction can be greater than the T95 boiling point for the lower boiling fraction of at least 5° F. (˜3° C.) greater, or at least 10° F. (˜6° C.) greater. In other additional or alternative aspects, initial/final and/or T5/T95 can be replaced with T2/T98 and/or T1/T99, as desired.

Alternatively, if a single condensation step occurs, rendering pyrolysis oil, the sugars may be isolated by adding enough water to induce phase separation. This can result in a separation of the anhydrosugars into the aqueous phase.

The aqueous anhydrosugar fractions, obtained from pyrolysis oil phase separation and/or from the staged condensation followed by phenolic oligomers removal, can contain further compounds, which can be removed to avoid inhibition/poisoning of the microorganisms downstream. Examples of suitable removal methods can include liquid-liquid extraction and/or solid-liquid extraction. In liquid-liquid extraction, the aqueous solution can be extracted with a non-miscible organic solvent, e.g., ethyl acetate and/or diethylether. Activated carbon can be an exemplary non-limiting solid for performing a solid-liquid extraction. Other approaches can include treatment of the aqueous anhydrosugar stream with microbial/enzymatic biocatalysts, e.g., biological abatement with fungi, and/or one or more chemical treatments. Overliming is a non-limiting example of a chemical treatment. Overliming describes the process of adding calcium hydroxide to the aqueous anhydrosugar stream. Without being bound by any particular theory, it is believed that overliming can cause a chemical transformation to occur causing undesired compounds to precipitate.

As an additional or alternative process for passivating the metals in a biomass feed prior to thermochemical conversion, instead of using an aqueous acid solution, an acid can be in a supercritical state, for example supercritical carbon dioxide (sc-CO₂). Lignocellulosic biomass can have increased solubility in sc-CO_{2,} allowing the acid to more easily penetrate the biomass structure and titrate metals, thus reducing the sizing requirements of the biomass. Further, after the pretreatment, the CO₂ can be removed by depressurizing the system. Use of sc-CO₂ can reduce and/or minimize the need for the energy intensive removal of water as well as the need for sizing of biomass to small dimensions. Use of sc-CO₂ as the solvent for delivering acid for metals passivation can also have further advantages over the pretreatment in aqueous acid solutions. As mentioned above, biomass solubility can be increased in sc-CO_{2.} Thus, the sc-CO₂ treatment can facilitate the breakdown of the lignocellulose structure. This effect can be increased after the treatment, when the pressure may be rapidly decreased, as in sc-CO₂ explosion treatment to remove the CO₂ from the biomass. The rapid expansion of the biomass can further break open the biomass structure. This can contribute to an increase in the concentration of levoglucosan and other desired products during pyrolysis, as mass transfer of these products from the biomass melt into the vapor phase can be facilitated and undesired side reactions can advantageously be reduced.

In various aspects, producing an anhydrosugar-containing feed where the presence of poisons is reduced and/or minimized (such as by the methods described above) can allow for an increase in conversion and/or conversion efficiency for anhydrosugars to fatty acid alkyl esters of at least about 5%, for example at least about 10%. Additionally or alternately, in various aspects, the genes for production of levoglucosan kinase and for production of fatty acid alkyl ester can be co-located on the same plasmid/vector/cassette and/or on the same chromosome (if the genetic modification is accomplished on a homologous or heterologous chromosome). Such co-location can facilitate co-expression, which can allow for an increase in conversion and/or conversion efficiency for anhydrosugars to fatty acid alkyl esters of at least about 5%, or at least about 10%.

After producing an anhydrosugar-containing feed, the feed can be used as part of a culturing environment for genetically modified E. coli. The culturing environment can include at least about 0.01 wt % of the genetically modified E. coli, for example at least about 0.05 wt %, at least about 0.1 wt %, at least about 0.5 wt %, or at least about 1 wt %. After conversion of anhydrosugars by the genetically modified E. coli, the culturing environment can further include at least about 0.01 wt % of a fatty acid alkyl ester produced by conversion of anhydrosugars by the genetically modified E. coli, for example at least about 0.05 wt %, at least about 0.1 wt %, at least about 0.5 wt %, or at least about 1 wt %.

### Pyrolysis of Biomass to Form Lignocellulose

In general, pyrolysis is a thermal degradation process in which large molecules are broken or cracked into smaller molecules in the presence of little, if any, oxygen. A wide variety of hydrocarbon materials can be pyrolyzed to produce vapor, liquid, and/or solid materials into more readily usable forms. Pyrolysis is generally distinct from other processes that use both heat and additional chemical reactivity to alter molecular structure, such as processes that usually take place in reactive (non-inert) atmospheres, e.g., hydroprocessing/hydrotreatment in the presence of hydrogen-containing gas, sulfiding in the presence of a sulfur-containing gas, and the like.

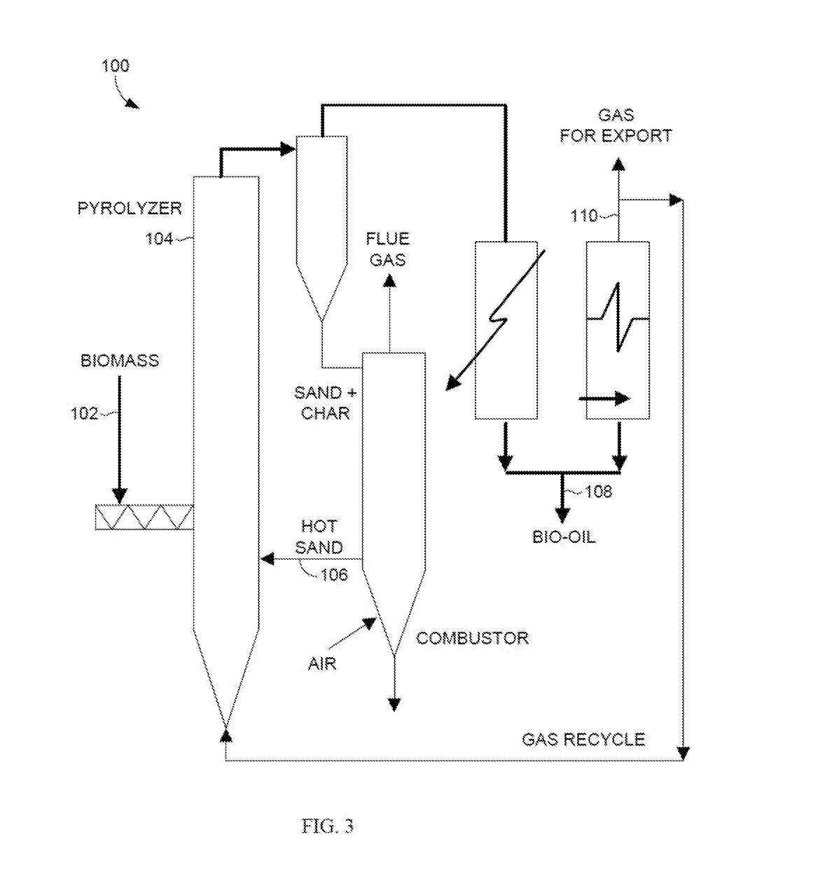

FIG. 3 schematically illustrates an example of a configuration 100 of a pyrolysis reactor suitable for producing pyrolysis bio-oil. As will be discussed in greater detail herein, bio-oil 108 can be produced from pyrolysis of biomass 102, such as but not limited to wood chips and/or corn stover. Depending on the source, bio-oil 108 can be a complex mixture of organic oxygenates, characterized by a relatively high oxygen content (e.g., >35%), reactive oxygen functional groups, thermal instability, corrosivity, low energy content, and a significant water fraction (10-20%), making it unsuitable for use as a refinery feedstock or transportation fuel without significant further upgrading. Bio-oil 108 can typically be produced using a fast pyrolysis process, where dry solid biomass can be converted to liquid products using a reactor with relatively high heat transfer rates, e.g., a fluidized bed reactor.

In a fast pyrolysis reactor, biomass 102 can be fed to a pyrolyzer 104, where it can be contacted with a circulating heat transfer medium, typically a fine, hot sand 106, resulting in high heating rates, on the order of 1000° C./sec. Average temperatures at the outlet of the pyrolyzer can be ˜500° C., with a typical residence time of less than two seconds for fast pyrolysis. The biomass 102 can undergo thermal depolymerization of the lignin/cellulose molecules, resulting in a complex mixture of oxygenated organics following rapid cooling. In addition to the bio-oil produced, a gas 110 (comprising predominately CO, CO₂, and H₂O) and char can be formed. The char can typically circulate with the sand back to the combustor where it can provide the heat required to bring the sand back to the desired temperature for the pyrolyzer 104.

In addition to levoglucosan, a bio-oil 108 produced via a fast pyrolysis reactor similar to the configuration shown in FIG. 3 can be a complex mixture of oxygenated organics typically comprising acids, aldehydes, ketones, phenolics, and alcohols. The composition can vary with biomass source and processing, among other variables.

In this discussion, the terms “pyrolyze” and “pyrolyzing” are considered to be the act of converting a compound by pyrolysis. Pyrolysis is considered to be a chemical process in which a feed material is converted to one or more products by heat. By this definition, reactions that occur by heating in the presence of substantially reactive compounds (e.g., oxygen, hydrogen, sulfur-containing gases, and the like, but not including catalysts) to cause any significant degree of reaction involving (e.g., oxidation of) the feed material, such as by side reactions, are substantially excluded. The terms “thermolysis” or “thermal reaction” are considered to be synonyms for the term pyrolysis. According to the present invention, the term “torrefaction” is also considered as being within the definition of pyrolysis.

A wide range of feedstocks of various types, sizes, and moisture contents can be processed according to aspects of the present invention. Feedstocks that can be used in aspects of the present invention can comprise any hydrocarbon that can be thermally decomposed and/or transformed. Preferably, the feedstock comprises biomass, particularly biomass not typically processed or easily processable through chemical reactions. For example, the feedstocks can be comprised of at least 10 wt % (for example at least 30 wt %, at least 50 wt %, at least 70 wt %, or at least 90 wt %) biomass, based on total weight of feedstock processed or supplied to the thermal or pyrolysis reactor.

The term “biomass,” for the purposes of the present invention, is considered any material not derived from fossil/mineral resources and comprising at least carbon, hydrogen, and oxygen. Examples of biomass can include, but are not limited to, plant and plant-derived material, algae and algae-derived material, vegetation, agricultural waste, forestry waste, wood waste, paper waste, animal-derived waste, poultry-derived waste, municipal solid waste, cellulose and cellulosics, carbohydrates or derivates thereof, charcoal, and the like, and combinations thereof. The feedstock can also comprise pyrolyzable components other than biomass, such as fossil/mineral fuels (e.g., coal, crude or refined petroleum feedstocks, and the like, as well as combinations thereof).

Additional or alternate examples of biomass feedstock components can include, but are not necessarily limited to, timber harvesting residues, softwood chips, hardwood chips, tree branches, tree stumps, leaves, bark, sawdust, off-spec paper pulp, corn, corn cob, corn stover, wheat straw, rice straw, sugarcane, bagasse, switchgrass, miscanthus, animal manure, municipal garbage, municipal sewage, commercial waste, grape pumice, almond shells, pecan shells, coconut shells, coffee grounds, grass pellets, hay pellets, wood pellets, cardboard, paper, plastic, cloth, and combinations thereof.

The biomass to be pyrolyzed may be ground prior to pyrolyzing. For example, the biomass can be ground in a mill until a desired particle size is achieved. In one embodiment, the particle size of the biomass to be pyrolyzed can be sufficient (with or without grinding) to pass through a 30 mm screen, for example a 20 mm screen, a 10 mm screen, a 5 mm screen, or a 1 mm screen.

Pyrolysis can preferably be carried out in the presence of little or no oxygen. If oxygen is present, it can be present in an amount less that the stoichiometric amount required for complete combustion. Preferably, pyrolysis can be carried out in an environment (e.g., in the pyrolysis reactor) having an oxygen content of less than 40%, for example less than 30%, less than 20%, less than 10%, less than 5%, less than 1%, less than 0.5%, or less than 0.01% of the stoichiometric amount of oxygen required for complete combustion of the feedstock. In another preferred embodiment, pyrolysis can be carried out in the absence of any added oxygen (in which case oxygen may be present in trace/contaminant amounts, but no oxygen is deliberately added).

In one embodiment, pyrolyzed product can exit the pyrolysis reactor in the vapor phase. In certain preferred embodiments, the vapor phase can be passed through a filter to separate any solids from the more desirable product. The filtered vapors can then be condensed to form one or more liquid products.

Condensation can be carried out using any equipment suitable for such purpose. For example, condensation can be carried out using a condensation train to collect the desired products. The condensation train can comprise at least one chilled water condenser, at least one electrostatic precipitator, or at least one coalescence filter, as well as combinations thereof.

The pyrolysis temperature can be sufficiently high to convert a sufficient quantity of feed to desired product, but not so high to produce undesired quantities of non-condensable gas or undesired solid. For example, feed can be pyrolyzed at a temperature from 200° C. to 600° C., for example from 300° C. to 600° C. or from 400° C. to 500° C.

The pyrolysis pressure can be within a range that reduces/minimizes formation of non-condensable gas and solid product. The pressure can range from about 0 psig (about 0 kPag) to about 1000 psig (about 6.9 MPag), for example from about 5 psig (about 35 kPag) to about 500 psig (about 3.5 MPag) or from about 10 psig (about 69 kPag) to about 200 psig (about 1.4 MPag).

Pyrolysis can generally be carried out for a time that enables a substantial quantity of feed to be converted into condensable vapor and/or liquid products. This can range over a wide period of time, depending upon pressure, temperature, and type of reactor used, inter alia. For example, pyrolysis can be carried out for a time from 0.1 second to 48 hours, for example from 0.1 second to 24 hours or from 0.1 second to 1 hour. Shorter times are generally more preferred, such as from 0.1 second to 1 minute or from 0.1 second to 10 seconds. Thus, in some embodiments, fast pyrolysis can be used. Fast pyrolysis is a high-temperature process in which feedstock is rapidly heated. In some embodiments, the feedstock can be heated in the substantial absence of oxygen. The feedstock can advantageously decompose to generate predominantly vapor and solid (char) products/by-products. The vapor product can preferably be cooled and condensed to form one or more liquid products. Multiple steps of heating and cooling can be carried out to produce intermediate pyrolysis liquid products. Fast pyrolysis processes can typically produce from about 60 wt % to about 75 wt % condensable gas and liquid products, from about 15 wt % to about 25 wt % solid char, and from about 10 wt % to about 20 wt % non-condensable gas products, but these relative numbers can depend heavily on the particular feedstock composition.

Slow pyrolysis can additionally or alternatively be used. In slow pyrolysis, the feedstock can preferably be heated to not greater than about 600° C. for a time period ranging from 1 minute to 24 hours, for instance from 1 minute to 1 hour. Vapor product typically does not escape as rapidly in slow pyrolysis as in fast pyrolysis. Thus, vapor products may react with each other as solid char and liquid are being formed. Rates of heating in slow pyrolysis can typically be slower than in fast pyrolysis. A feedstock can be held at constant temperature or slowly heated. Vapors can be continuously removed as they are formed.

Vacuum pyrolysis can additionally or alternately be used. In vacuum pyrolysis, the feedstock is maintained at less than atmospheric pressure (i.e., below 0 psig or 0 kPag, but above 0 psia or 0 kPaa). Vacuum conditions can be used to decrease the boiling point, to avoid adverse chemical reactions, and/or to reduce the heating duty by using relatively lower temperatures.

Pyrolysis product can contain water. As an example, condensed pyrolysis product can contain from 10 wt % to 30 wt % water. If desired, the water can be removed using any appropriate means, such as by flashing, decanting, distillation, membrane separation, or the like, or any combination thereof.

The pyrolysis can be performed in the presence of particulates (such as sand) that can assist with heat control. Alternatively, an upgraded pyrolysis product can be formed by using a catalyst during pyrolysis comprising a hydrogenation metal on a basic metal oxide support. Such catalysts are described in U.S. Patent Application Publication No. 2015/0321980, which is incorporated herein by reference.

As pyrolysis products leave the reactor, they can be in the form of vapor, liquid, and/or solid. A substantial portion of the vapor can preferably be a condensable vapor, e.g., a condensable C₃⁺ hydrocarbon. In a preferred embodiment, a substantial portion of the vapor exiting the reactor can be condensed to form a fuel or pyrolysis oil. These various products can be isolated by way of a condenser system. The products can be used as fuels and/or as a variety of chemicals.

Modification of E. coli for Levoglucosan Kinase Production and Fatty Acid Alkyl Ester Production

Modification of bacteria (or other microbes) for insertion of desirable genetic sequences is a known process. Any convenient conventional method for introduction of genetic sequences into E. coli, or another suitable production host, can be used.

The production host used to produce fatty acid alkyl (e.g., methyl or ethyl) esters from levoglucosan can be recombinantly modified to include nucleic acid sequences that express or over-express peptides. The modifications to the production host described herein can be through genomic alterations, addition of recombinant expression systems, or combinations thereof. For each of the modifications described herein, the nucleic acid sequences can be native or non-native. For each of the modifications described herein, the modification can include genetic modification with a construct that includes a gene (i.e., a nucleic acid sequence complete enough to encode a desired function) and a corresponding promoter for expression of the gene. For each of the modifications described herein, the corresponding promoters for the nucleic acid sequences can be constitutive or inducible. For each of the modifications described herein, the corresponding promoters for the nucleic acid sequences can be homologous or heterologous, but typically can be heterologous (even if otherwise native).

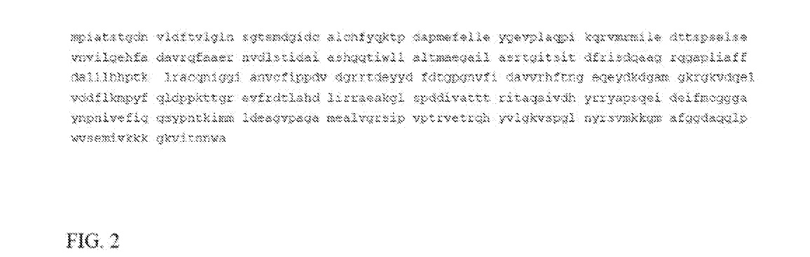

Levoglucosan can be converted to glucose using the enzyme levoglucosan kinase (such as EC 6.2.1.-), which is shown in FIG. 2. This enzyme can phosphorylate levoglucosan to form glucose-6-phosphate. Glucose-6-phosphate is readily fermentable by substantially all microorganisms without further manipulation. Genes that encode for the levoglucosan kinase enzyme shown in FIG. 2 are present in several microorganisms, such as Aspergillus niger, Lipomyces starkeyi, and Rhodosporidium toruloides. Introduction of genes that encode for the levoglucosan kinase enzyme have previously been introduced into E. coli. See, for example, Dai et al., World Journal of Microbiology and Biotechnology 25:9, 2009; and/or Lian et al., Bioresource Technology 133, 2013.

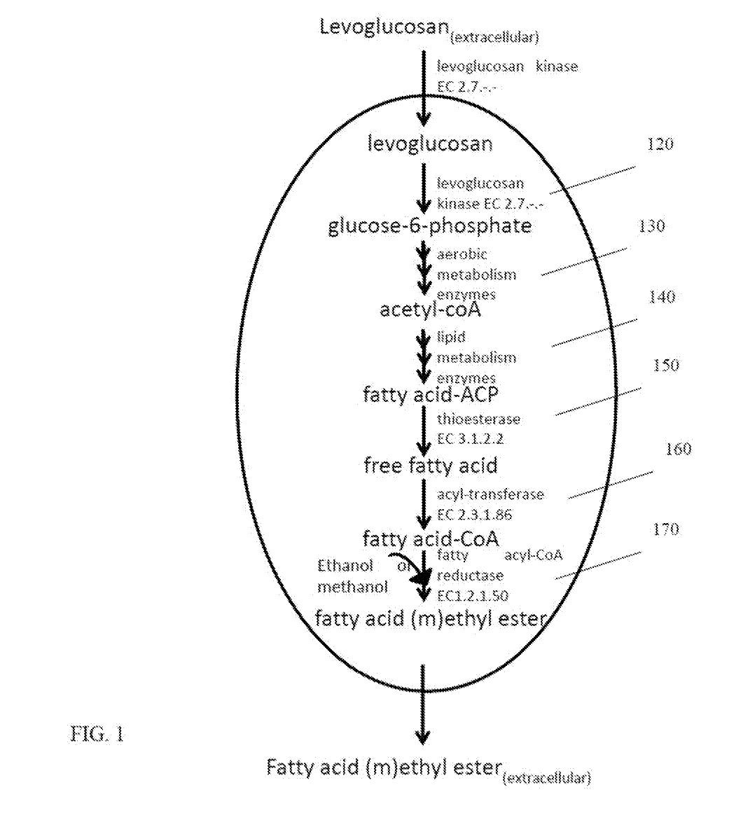

After conversion of levoglucosan to glucose-6-phosphate, the E. coli can ferment the glucose to form fatty acid alkyl esters, such as fatty acid methyl esters or fatty acid ethyl esters. FIG. 1 shows an example of a fermentation pathway for a genetically modified E. coli. By modifying an E. coli to include genes that encode for levoglucosan kinase, an E. coli can be modified to perform the processes shown in FIG. 1. Such an E. coli can use levoglucosan as an initial carbon source and can (internally) produce a fatty acid alky ester product.

In addition to introducing the gene for levoglucosan kinase enzyme 120, such as the sequence shown in FIG. 2, an E. coli can be modified to express and/or enhance the expression of other enzymes shown in FIG. 1. The additional enzymes can include aerobic metabolism enzymes 130 for conversion of glucose to acetyl-coenzyme A, or acetyl-CoA; lipid metabolism enzymes 140 for conversion of acetyl-CoA to fatty acid acyl carry protein, or fatty acid ACP; thioesterase 150 for conversion of fatty acid ACP to a free fatty acid; acyl transferase enzymes 160 for conversion of free fatty acid to fatty acid-CoA; and fatty acyl-CoA reductase enzymes 170 for conversion, with a source of small chain alcohol, for fatty acid-CoA to fatty acid alkyl ester.

As noted above, the aerobic metabolism enzymes 130 for conversion of glucose and/or glucose-6-phosphate are typically present in a wide variety of microorganisms, including E. coli. Optionally, production of these enzymes can be enhanced by genetic modification and/or production of enzymes for pathways that compete with production of acetyl-CoA could be reduced.

Lipid metabolism enzymes 140 correspond to enzymes for converting acetyl-CoA to longer chain fatty acid type compounds. Examples of such enzymes can include those used in the fatty acid synthase (FAS) pathway. Fatty acid synthase (FAS) is a group of peptides that catalyze the initiation and elongation of acyl chains (Marrakchi et al., Biochemical Society, 30:1050-1055, 2002). The acyl carrier protein (ACP) along with the enzymes in the FAS pathway control the length, degree of saturation, and branching of the fatty acids produced.

One example of a fatty acid biosynthetic pathway based on FAS can be a pathway that uses the precursors acetyl-CoA and malonyl-CoA. The steps in this type of pathway can be catalyzed, for example, by enzymes of the fatty acid biosynthesis (fab) and acetyl-CoA carboxylase (acc) gene families. This pathway is described in Heath et al., Prog. Lipid Res. 40(6):467-97 (2001).

Acetyl-CoA can be carboxylated by acetyl-CoA carboxylase (Acc, a multisubunit enzyme encoded by four separate genes, accABCD), to form malonyl-CoA. The malonate group can be transferred to ACP by malonyl-CoA:ACP transacylase (FabD) to form malonyl-ACP. A condensation reaction can then be used to combine malonyl-ACP with acetyl-CoA to form β-ketoacyl-ACP. β-ketoacyl-ACP synthase III (FabH) can then initiate the FAS cycle.

A cycle of steps can then be repeated until a saturated fatty acid of a desired length is made. First, 3-ketoacyl-ACP can be reduced by NADPH to form β-hydroxyacyl-ACP. This step can be catalyzed by β-ketoacyl-ACP reductase (FabG). β-hydroxyacyl-ACP can then be dehydrated to form trans-2-enoyl-ACP. β-hydroxyacyl-ACP dehydratase/isomerase (FabA) or β-hydroxyacyl-ACP dehydratase (FabZ) can catalyze this step. NADPH-dependent trans-2-enoyl-ACP reductase I, II, or III can reduce trans-2-enoyl-ACP to form acyl-ACP. Subsequent cycles can be started by the condensation of malonyl-ACP with acyl-ACP by β-ketoacyl-ACP synthase I (FabB) or β-ketoacyl-ACP synthase II (FabF).

The above pathway provides one example of how lipid metabolism enzymes as part of the FAS pathway can be used to build a fatty acid-type chain within an E. coli in the form of a fatty acid-ACP. Optionally, further modifications can be made to an E. coli to alter the production of various products or enzymes. This can include enhancing production of desired products and/or desired enzymes as well as reducing production of competing products and/or competing enzymes.

Control over production of fatty acids generally, or over production of fatty acids with a desired chain length, can be provided by controlling the expression of thioesterase enzyme 150 (EC 3.1.2.2). Conventional microorganism fermentation pathways, such as the pathways in a conventional E. coli, can involve a balance between expression of the enzyme sn-glycerol-3-phosphate acyltransferase (PlsB) and various thioesterase enzymes. Inhibiting PlsB can lead to an increase in the levels of long chain acyl-ACP. Thioesterase overexpression can be lead to increased fatty acid production. In combination, control over expression of these enzymes can generally allow for increased fatty acid production.

If specific chain lengths are desired for fatty acids, additional control can be provided by controlling the expression of thioesterases specific to various carbon chain lengths for a fatty acid, such as fatty acid carbon chain lengths from 10 to 22. This can include increasing the expression of thioesterase for a desired chain length and/or decreasing expression of non-desired chain lengths.

After forming fatty acids, the fatty acids can be converted to a fatty acid-CoA using acyl transferase enzymes 160 (EC 2.3.1.86). For example, acyl-CoA synthase (ACS) can esterify free fatty acids to a fatty acid-CoA by a two-step mechanism. A fatty acid can first be converted to an acyl-AMP intermediate (an adenylate) through the pyrophosphorolysis of ATP. The activated carbonyl carbon of the adenylate can then be coupled to the thiol group of CoA, releasing AMP and the acyl-CoA final product. See Shockey et al., Plant. Physiol. 129:1710-1722, 2002.

Fatty acid-CoA can then be converted into fatty acid alkyl esters, such as fatty acid methyl esters, fatty acid ethyl esters, or another suitable fatty acid short chain ester. This conversion can be performed by using an E coli optionally genetically modified to include fatty acyl-CoA reductase 170 (EC 1.2.1.50). The type of fatty acid ester formed can be controlled based on the type of short chain alcohol available for use. The production of fatty esters, including waxes, from acyl-CoA and alcohols, can be engineered using known polypeptides. One method of making fatty esters includes increasing the expression of, or expressing more active forms of, one or more ester synthases (EC 2.3.1.20, 2.3.1.75). Ester synthase peptide sequences are publicly available. Optionally, an ester exporter such as a member of the FATP family can be used to facilitate the release of esters into the extracellular environment. A non-limiting example of an ester exporter that can be used is fatty acid (long chain) transport protein CG7400-PA, isoform A, from Drosophila melanogaster, at locus NP. sub.-524723.

### Additional Embodiments

Embodiment 1. A method for converting levoglucosan to fatty acid alkyl esters, comprising: pyrolyzing a biomass feed under effective pyrolysis conditions to form a pyrolysis product comprising levoglucosan; performing a separation on at least a portion of the pyrolysis product to form a levoglucosan-enriched product; and culturing a recombinant Escherichia coli cell in the levoglucosan-enriched product to form a fermentation product comprising a fatty acid alkyl ester, the recombinant Escherichia coli cell comprising an expressed gene encoding a levoglucosan kinase enzyme and at least one expressed gene encoding a fatty acid derivative enzyme for production of the fatty acid alkyl ester internal to the Escherichia coli cell.

Embodiment 2. The method of Embodiment 1, further comprising treating the biomass feed with an acid prior to pyrolyzing under effective conditions to passivate metals in the biomass feed.

Embodiment 3. The method of Embodiment 2, wherein the biomass feed is treated with a solution of an acid in supercritical CO₂.

Embodiment 4. The method of Embodiment 3, wherein the treating of the biomass feed with a solution of an acid in supercritical CO₂ increases conversion and/or conversion efficiency for anhydrosugars to fatty acid alkyl esters by at least about 5%.

Embodiment 5. The method of any of the above embodiments, wherein the biomass feed is pyrolyzed under effective fast pyrolysis conditions.

Embodiment 6. The method of any of the above embodiments, wherein forming a pyrolysis product comprises staging condensation of a pyrolysis effluent to form plurality of pyrolysis effluent fractions, the pyrolysis product comprising at least a portion of a pyrolysis effluent fraction.

Embodiment 7. The method of any of the above embodiments, wherein performing a separation on at least a portion of the pyrolysis product comprises washing the at least a portion of the pyrolysis product with water; and performing at least one of filtration or centrifugation on the washed pyrolysis product to form a filtered washed pyrolysis product comprising the levoglucosan-enriched product.

Embodiment 8. The method of any of the above embodiments, wherein performing a separation on at least a portion of the pyrolysis product comprises performing a solid-liquid extraction, a liquid-liquid extraction, or a combination thereof on at least one of the washed pyrolysis product, the filtered washed pyrolysis product, and the at least a portion of the pyrolysis product to form the levoglucosan-enriched product.

Embodiment 9. The method of any of the above embodiments, wherein performing a separation on at least a portion of the pyrolysis product comprises chemically treating at least one of the washed pyrolysis product, the filtered washed pyrolysis product, and the at least a portion of the pyrolysis product to form a product comprising the levoglucosan-enriched product, the chemically treating optionally comprising overliming.

Embodiment 10. The method of any of the above embodiments, wherein performing a separation on at least a portion of the pyrolysis product comprises treating, with a microbial and/or enzymatic biocatalyst, at least one of the washed pyrolysis product, the filtered washed pyrolysis product, and the at least a portion of the pyrolysis product to form a product comprising the levoglucosan-enriched product.

Embodiment 11. The method of any of the above embodiments, wherein performing a separation on at least a portion of the pyrolysis product increases conversion and/or conversion efficiency for anhydrosugars to fatty acid alkyl esters by at least about 5%.

Embodiment 12. The method of any of the above embodiments, wherein the levoglucosan-enriched product further comprises a 5-carbon anhydrosugar.

Embodiment 13. A cultured recombinant Escherichia coli cell, said cell comprising: at least one expressed nucleic acid, operably linked to a first promoter that is constitutive, encoding an enzyme comprising an acyl-CoA synthase; at least one expressed nucleic acid, operably linked to a second promoter that is constitutive, encoding an enzyme comprising a thioesterase; at least one expressed nucleic acid, operably linked to a third promoter that is constitutive, encoding an enzyme comprising a fatty acyl-CoA reductase; and an expressed non-native nucleic acid, operably linked to a fourth promoter that is constitutive, encoding a levoglucosan kinase enzyme, wherein, when cultured in the presence of a carbon source comprising levoglucosan, said cultured recombinant cell produces a fatty acid alkyl ester.

Embodiment 14. The cultured recombinant Escherichia coli cell of Embodiment 13, wherein the fatty acid alkyl ester comprises a fatty acid methyl ester, a fatty acid ethyl ester, or a combination thereof.

Embodiment 15. The cultured recombinant Escherichia coli cell of any of Embodiments 13-14, wherein the fatty acid alkyl ester comprises a fatty acid carbon chain length of 10 carbons to 22 carbons.

Embodiment 16. The cultured recombinant Escherichia coli cell of any of Embodiments 13-15, wherein the fourth promoter is a heterologous promoter.

Embodiment 17. The cultured recombinant Escherichia coli cell of any of Embodiments 13-16, wherein at least one of the nucleic acid encoding an enzyme comprising an acyl-CoA synthase, the nucleic acid encoding an enzyme comprising a thioesterase, and the nucleic acid encoding an enzyme comprising a fatty acyl-CoA reductase, comprises a non-native nucleic acid.

Embodiment 18. The cultured recombinant Escherichia coli cell of any of Embodiments 13-17, wherein at least one of the first promoter, the second promoter, and the third promoter is a heterologous promoter.

Embodiment 19. The cultured recombinant Escherichia coli cell of any of Embodiments 13-18, wherein the non-native nucleic acid encoding levoglucosan kinase enzyme and the nucleic acid encoding an enzyme comprising a fatty acyl-CoA reductase are located on the same plasmid, an amount of conversion of levoglucosan to the fatty acid alkyl ester being at least about 5% greater than an amount of conversion for a recombinant Escherichia coli cell where the non-native nucleic acid encoding levoglucosan kinase enzyme and the nucleic acid encoding an enzyme comprising a fatty acyl-CoA reductase are located on different plasmids.

Embodiment 20. A cell culturing environment comprising: at least about 0.1 wt % of a fatty acid alkyl ester; and at least about 0.1 wt % of cultured recombinant Escherichia coli cells, the cultured recombinant Escherichia coli cells comprising: at least one expressed nucleic acid, operably linked to a first promoter that is constitutive, encoding an enzyme comprising an acyl-CoA synthase; at least one expressed nucleic acid, operably linked to a second promoter that is constitutive, encoding an enzyme comprising a thioesterase; at least one expressed nucleic acid, operably linked to a third promoter that is constitutive, encoding an enzyme comprising a fatty acyl-CoA reductase; and an expressed non-native nucleic acid, operably linked to a fourth promoter that is constitutive, encoding a levoglucosan kinase enzyme, wherein the fatty acid alkyl ester comprises fatty acid alkyl ester produced by conversion of anhydrosugars by the cultured recombinant Escherichia coli cells.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

1. A method for converting levoglucosan to fatty acid alkyl esters, comprising:
pyrolyzing a biomass feed under effective pyrolysis conditions to form a pyrolysis product comprising levoglucosan;
performing a separation on at least a portion of the pyrolysis product to form a levoglucosan-enriched product; and
culturing a recombinant Escherichia coli cell in the levoglucosan-enriched product to form a fermentation product comprising a fatty acid alkyl ester, the recombinant Escherichia coli cell comprising an expressed gene encoding a levoglucosan kinase enzyme and at least one expressed gene encoding a fatty acid derivative enzyme for production of the fatty acid alkyl ester internal to the Escherichia coli cell.

2. The method of claim 1, further comprising treating the biomass feed with an acid prior to pyrolyzing under effective conditions to passivate metals in the biomass feed.

3. The method of claim 2, wherein the biomass feed is treated with a solution of an acid in supercritical CO₂.

4. The method of claim 3, wherein the treating of the biomass feed with a solution of an acid in supercritical CO₂ increases conversion and/or conversion efficiency for anhydrosugars to fatty acid alkyl esters by at least about 5%.

5. The method of claim 1, wherein the biomass feed is pyrolyzed under effective fast pyrolysis conditions.

6. The method of claim 1, wherein forming a pyrolysis product comprises staging condensation of a pyrolysis effluent to form plurality of pyrolysis effluent fractions, the pyrolysis product comprising at least a portion of a pyrolysis effluent fraction.

7. The method of claim 1, wherein performing a separation on at least a portion of the pyrolysis product comprises washing the at least a portion of the pyrolysis product with water, and performing at least one of filtration or centrifugation on the washed pyrolysis product to form a filtered washed pyrolysis product comprising the levoglucosan-enriched product.

8. The method of claim 1, wherein performing a separation on at least a portion of the pyrolysis product comprises performing a solid-liquid extraction, a liquid-liquid extraction, or a combination thereof on at least one of the washed pyrolysis product, the filtered washed pyrolysis product, and the at least a portion of the pyrolysis product to form the levoglucosan-enriched product.

9. The method of claim 1, wherein performing a separation on at least a portion of the pyrolysis product comprises chemically treating at least one of the washed pyrolysis product, the filtered washed pyrolysis product, and the at least a portion of the pyrolysis product to form a product comprising the levoglucosan-enriched product, the chemically treating optionally comprising overliming.

10. The method of claim 1, wherein performing a separation on at least a portion of the pyrolysis product comprises treating, with a microbial and/or enzymatic biocatalyst, at least one of the washed pyrolysis product, the filtered washed pyrolysis product, and the at least a portion of the pyrolysis product to form a product comprising the levoglucosan-enriched product.

11. The method of claim 1, wherein performing a separation on at least a portion of the pyrolysis product increases conversion and/or conversion efficiency for anhydrosugars to fatty acid alkyl esters by at least about 5%.

12. The method of claim 1, wherein the levoglucosan-enriched product further comprises a 5-carbon anhydrosugar.

13. A cultured recombinant Escherichia coli cell, said cell comprising:
at least one expressed nucleic acid, operably linked to a first promoter that is constitutive, encoding an enzyme comprising an acyl-CoA synthase;
at least one expressed nucleic acid, operably linked to a second promoter that is constitutive, encoding an enzyme comprising a thioesterase;
at least one expressed nucleic acid, operably linked to a third promoter that is constitutive, encoding an enzyme comprising a fatty acyl-CoA reductase; and
an expressed non-native nucleic acid, operably linked to a fourth promoter that is constitutive, encoding a levoglucosan kinase enzyme,
wherein, when cultured in the presence of a carbon source comprising levoglucosan, said cultured recombinant cell produces a fatty acid alkyl ester.

14. The cultured recombinant Escherichia coli cell of claim 13, wherein the fatty acid alkyl ester comprises a fatty acid methyl ester, a fatty acid ethyl ester, or a combination thereof.

15. The cultured recombinant Escherichia coli cell of claim 13, wherein the fatty acid alkyl ester comprises a fatty acid carbon chain length of 10 carbons to 22 carbons.

16. The cultured recombinant Escherichia coli cell of claim 13, wherein the fourth promoter is a heterologous promoter.

17. The cultured recombinant Escherichia coli cell of claim 13, wherein at least one of the nucleic acid encoding an enzyme comprising an acyl-CoA synthase, the nucleic acid encoding an enzyme comprising a thioesterase, and the nucleic acid encoding an enzyme comprising a fatty acyl-CoA reductase comprises a non-native nucleic acid.

18. The cultured recombinant Escherichia coli cell of claim 13, wherein at least one of the first promoter, the second promoter, and the third promoter is a heterologous promoter.

19. The cultured recombinant Escherichia coli cell of claim 13, wherein the non-native nucleic acid encoding levoglucosan kinase enzyme and the nucleic acid encoding an enzyme comprising a fatty acyl-CoA reductase are located on the same plasmid, an amount of conversion of levoglucosan to the fatty acid alkyl ester being at least about 5% greater than an amount of conversion for a recombinant Escherichia coli cell where the non-native nucleic acid encoding levoglucosan kinase enzyme and the nucleic acid encoding an enzyme comprising a fatty acyl-CoA reductase are located on different plasmids.

20. A cell culturing environment comprising:
at least about 0.1 wt % of a fatty acid alkyl ester; and
at least about 0.1 wt % of cultured recombinant Escherichia coli cells, the cultured recombinant Escherichia coli cells comprising:
at least one expressed nucleic acid, operably linked to a first promoter that is constitutive, encoding an enzyme comprising an acyl-CoA synthase;
at least one expressed nucleic acid, operably linked to a second promoter that is constitutive, encoding an enzyme comprising a thioesterase;
at least one expressed nucleic acid, operably linked to a third promoter that is constitutive, encoding an enzyme comprising a fatty acyl-CoA reductase; and
an expressed non-native nucleic acid, operably linked to a fourth promoter that is constitutive, encoding a levoglucosan kinase enzyme,
wherein the fatty acid alkyl ester comprises fatty acid alkyl ester produced by conversion of anhydrosugars by the cultured recombinant Escherichia coli cells.


SEQUENCE LISTING (1 sequence)

SEQ ID NO: 1 - protein, 419 aa, Lipomyces starkeyi
MPIATSTGDNVLDFTVLGLNSGTSMDGIDCALCHFYQKTPDAPMEFELLEYGEVPLAQPIKQRVMRMILEDTTSPSELSEVNVILGEHFADAVRQFAAERNVDLSTIDAIASHGQTIWLLALTMAEGAILASRTGITSITDFRISDQAAGRQGAPLIAFFDALLLHHPTKLRACQNIGGIANVCFIPPDVDGRRTDEYYDFDTGPGNVFIDAVVRHFTNGEQEYDKDGAMGKRGKVDQELVDDFLKMPYFQLDPPKTTGREVFRDTLAHDLIRRAEAKGLSPDDIVATTTRITAQAIVDHYRRYAPSQEIDEIFMCGGGAYNPNIVEFIQQSYPNTKIMMLDEAGVPAGAMEALVGRSIPVPTRVETRQHYVLGKVSPGLNYRSVMKKGMAFGGDAQQLPWVSEMIVKKKGKVITNNWA